(19) United States
(12) Patent Application Publication
(10) Pub. No.: US 2026/0096796 A1
(43) Pub. Date: Apr. 9, 2026

(54) SYSTEMS AND METHODS OF FEATURE DETECTION WITHIN MEDICAL IMAGES

(71) Applicant: University of Louisville Research Foundation, Inc., Louisville, KY (US)

(72) Inventors: Aly Farag, Louisville, KY (US); Mohamed Yousuf, Louisville, KY (US); Samir Harb, Louisville, KY (US); Asem Ali, Louisville, KY (US)

(21) Appl. No.: 19/353,005

(22) Filed: Oct. 8, 2025

Related U.S. Application Data
(60) Provisional application No. 63/704,780, filed on Oct. 8, 2024.

(51) Int. Cl. A61B 6/00; A61B 6/03; A61B 6/50; G06T 7/00; G06T 7/11; G06V 10/26; G06V 10/764; G06V 10/77; G06V 10/82; G16H 30/20

(52) U.S. Cl. CPC A61B 6/5217; A61B 6/032; A61B 6/50; A61B 6/5294; G06T 7/0012; G06T 7/11; G16H 30/20; G06T 2207/10081; G06T 2207/20081; G06T 2207/20084; G06T 2207/30032; G06V 10/26; G06V 10/764; G06V 10/7715; G06V 10/82; G06V 2201/031

(57) ABSTRACT

A method including receiving image data including a plurality of CT scan images of at least a portion of a subject; segmenting the CT scan images to identify portions of each image corresponding to the subject's colon; analyze axial views of the segmented CT scan images to identify a candidate polyp using a first CNN; analyzing at least two of axial views, sagittal views, and coronal views CT scan images corresponding to the candidate polyp using a second model to classify the candidate polyp as a polyp or not a polyp; and generating a user interface that includes the classified candidate polyp.

### CROSS-REFERENCE TO RELATED APPLICATIONS

This application claims the benefit of U.S. Provisional Patent Application No. 63/704,780, filed on Oct. 8, 2024, the entire contents of which are incorporated herein by reference.

### STATEMENT REGARDING FEDERALLY SPONSORED RESEARCH OR DEVELOPMENT

This invention was made with government support under award numbers (i) 1602333 and (ii) 2124316 awarded by the National Science Foundation, as well as award numbers (i) 1R43CA179911-01 and (ii) 1R43CA250750-01 awarded by the National Institutes of Health. The government has certain rights in the invention.

### BACKGROUND

The present disclosure relates generally to the field of medical imaging, and more specifically to systems and methods of feature detection within medical images such as CT scan images.

### SUMMARY

In some aspects, the techniques described herein relate to a method for colon polyp detection, the method including: determining, by a first classifier, wherein the first classifier includes a first CNN model configured to use a first set of medical images including segmented medical images of a colon, a candidate polyp location and size; and classifying, by a second classifier, wherein the second classifier includes a second CNN model configured use a second set of medical images of the colon, a candidate polyp as a polyp or not a polyp; wherein the first classifier and the second classifier are configured in a cascade.

In some aspects, the techniques described herein relate to a method, wherein the first CNN model is trained on the first set of medical images of the colon. In some aspects, the techniques described herein relate to a method, wherein the first set of medical images includes segmented CT scans in an axial view of the colon. In some aspects, the techniques described herein relate to a method, wherein the second CNN model is trained on a second set of medical images of the colon. In some aspects, the techniques described herein relate to a method, wherein the second set of medical images includes 2D images in an axial view, a sagittal view, and a coronal view of the colon. In some aspects, the techniques described herein relate to a method, wherein the second CNN model is trained independently on each of the axial view, sagittal view, and coronal view of the 2D images. In some aspects, the techniques described herein relate to a method, wherein the second CNN model is configured to provide a classification and a weight related to each of the axial view, sagittal view, and coronal view of the 2D images. In some aspects, the techniques described herein relate to a method, wherein the trained second CNN model is configured to select and output a classification prediction based on the classification and the weight related to each of the axial view, sagittal view, and coronal view of the 2D images. In some aspects, the techniques described herein relate to a method, wherein the second set of medical images include DICOM data. In some aspects, the techniques described herein relate to a method, wherein the first classifier is configured to predict all true positive candidate polyps and a number of false positives candidate polyps. In some aspects, the techniques described herein relate to a method, wherein the second classifier is applied to each candidate polyp location and size. In some aspects, the techniques described herein relate to a method, wherein the first CNN model and the second CNN model are 2D CNN networks.

In some aspects, the techniques described herein relate to a system for colon polyp detection, the system including a processor and a memory, the memory storing instructions thereon, that when executed by the processor, cause the processor to: receive a set of CT scans, wherein a colon is segmented from the set of CT scans; construct a 3D model of the colon from the set of CT scans; simulate an inner wall of a region of interest of the colon; generate 2D images of the simulated inner wall; generate a plurality of feature maps using the 2D images and the 3D model; and detect, by a convolutional neural network (CNN) model using the plurality of feature maps, a candidate polyp location and size. In some aspects, the techniques described herein relate to a system, wherein detecting the candidate polyp location and size includes detecting the candidate polyp location and size by the CNN model.

In some aspects, the techniques described herein relate to a system, wherein the plurality of feature maps encode 3D surface geometry. In some aspects, the techniques described herein relate to a system, wherein the plurality of feature maps are combined as multi-channel images and provided to the CNN model. In some aspects, the techniques described herein relate to a system, wherein the CNN model is trained and validated using the plurality of feature maps. In some aspects, the techniques described herein relate to a system, wherein the plurality of feature maps include a depth map, a normal map, and a curvature map. In some aspects, the techniques described herein relate to a system, wherein the curvature map includes a curvature, wherein the curvature is calculated using moving least squares (MLS) fitting algebraic spheres to a surface of the 3D model of the colon.

In some aspects, the techniques described herein relate to a non-transitory computer-readable storage medium, having instructions stored thereon, that, when executed by a processor, cause the processor to: receive image data including a plurality of CT scan images of at least a portion of a subject; segment the CT scan images to identify portions of each image corresponding to the subject's colon; analyze axial views of the segmented CT scan images to identify a candidate polyp using a first CNN; analyze at least two of axial views, sagittal views, and coronal views CT scan images corresponding to the candidate polyp using a second model to classify the candidate polyp as a polyp or not a polyp; and generate a user interface that includes the classified candidate polyp.

### BRIEF DESCRIPTION OF THE DRAWINGS

The above and other aspects and features of the present disclosure will become more apparent to those skilled in the art from the following detailed description of the example embodiments with reference to the accompanying drawings.

FIG. 1 is a diagram illustrating a colonography method, according to an exemplary embodiment.

FIG. 2 is a block diagram of a computing system for segmenting medical images and analyzing the medical images to identify one or more features, according to an exemplary embodiment.

FIGS. 3A-B are block diagrams of a segmentation/modeling circuit of the computing system of FIG. 2, according to several exemplary embodiments.

FIG. 4 is a flow diagram illustrating a method of segmenting/modeling a region of interest, such as a colon, according to an exemplary embodiment.

FIGS. 5A-B illustrate virtual images generated by the system of FIG. 2, according to an exemplary embodiment.

FIG. 6A illustrates an example camera geometry for visualization of a generated model.

FIG. 6B illustrates an example configuration of a ring of virtual cameras for generating a visualization.

FIG. 6C illustrates an example visualization/user interface component.

FIG. 7 illustrates an example user interface for visualizing/surfacing information for a medical professional.

FIGS. 8A-B are block diagrams of an analysis circuit of the computing system of FIG. 2, according to several exemplary embodiments.

### DETAILED DESCRIPTION

Referring generally to the FIGURES, described herein are systems and methods of feature detection (e.g., polyp detection) within medical images such as CT scan images.

In some contexts, it may be beneficial or desirable to detect one or more features within medical imaging data. For example, it may be beneficial to automatically analyze CT scan images to detect polyps in image data of a subject's colon. Conventional feature detection methods, such as optical colonoscopy, may have drawbacks such as cost, time, and invasiveness. Similarly, in some contexts, conventional computed tomography colonography (CTC) may have drawbacks such as lower accuracy and/or higher computational costs (e.g., processing power required, processing time required, etc.). Systems and methods of the present disclosure may overcome one or more of these drawbacks by generating a 3D model of an ROI (e.g., a subject's colon), generating one or more visualizations of the ROI, and/or analyzing the ROI to detect one or more features (e.g., polyps, etc.) in a manner that reduces computational costs (e.g., by using a 2D CNN rather than a 3D CNN, by using fewer parameters for inference, etc.), increases accuracy (e.g., by combining 2D and 3D feature information, etc.), increases the speed at which features can be detected (e.g., by reducing the need for invasive procedures and prep, by reducing processing time, etc.), and reduces the invasiveness of feature detection.

In various embodiments, systems and methods of the present disclosure offer one or more benefits. For example, systems and methods of the present disclosure may (i) facilitate improved generation of 3D models of a region of interest (e.g., a colon, etc.) based on image data, (ii) facilitate improved detection of features such as colorectal polyps, (iii) reduce an amount of computation required to automatically detect features based on image data (e.g., by using a 2D model neural network rather than a 3D neural network, by using fewer parameters, etc.), (iv) reduce a need for large datasets (e.g., manually annotated datasets, etc.) for training feature detection models, (v) improve identification of unfamiliar object classes (e.g., polyp-like objects, etc.), (vi)

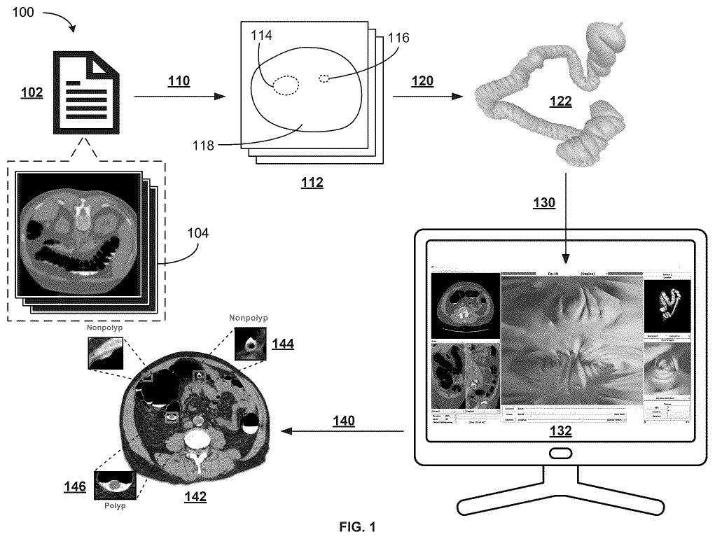

Referring now to FIG. 1, a colonography method (shown as method 100) is shown, according to an exemplary embodiment. In various embodiments, method 100 relates to a computed tomographic colonography (CTC) platform. CTC may include (i) image segmentation (e.g., to isolate lumen from other tissue, to address imaging uncertainties, etc.), (ii) 3D model generation to generate a colon model and register image data, (iii) visualization to display lumen on radiology stations (e.g., with details in 3D and corresponding 2D CT, etc.) and facilitate polyp editing, and (iv) analysis to detect polyps, classify detected polyps, and archive results in a patient record. In various embodiments, 3D model generation includes determining a centerline of the colon.

In various embodiments, method 100 begins with image data (shown as file 102). File 102 may be and/or include a DICOM file generated from a CT scan of a subject's abdomen. In various embodiments, file 102 includes 2D and/or 3D information. For example, file 102 may include volumetric information of a subject's colon and/or may include one or more 2D images (shown as images 104) of a subject's colon (e.g., a sagittal view, an axial view, and/or a coronal view, etc.).

At step 110, method 100 may include segmenting images 104 to isolate regions within the images and generate segmented images 112. For example, step 110 may include isolating lumen from other abdomen tissue (e.g., liver, lungs, small intestine, etc.). In various embodiments, step 110 identifies one or more regions within images 104. For example, step 110 may identify first colon region 114, second colon region 116, and non-colon region 118. At step 120, method 100 may include generating a 3D model (shown as model 122) based on segmented images 112. For example, method 100 may generate a 3D model of a subject's colon based on segmented CT scan images. In some embodiments, step 120 includes identifying a centerline of the structure in the model (e.g., a centerline of a subject's colon to facilitate a fly-through visualization, etc.).

At step 130, method 100 may include generating one or more visualizations based on model 122. For example, method 100 may include generating a display or dashboard (shown as display 132) to facilitate review by a medical professional. Display 132 may include one or more views of model 122 and/or augmented image data. At step 140, method 100 may include analyzing model 122 to identify one or more features within model 122. For example, method 100 may identify polyps within a model of a subject's colon. In various embodiments, step 140 includes surfacing this information to a medical professional via a user interface (e.g., shown as user interface 142 including identified polyp 146 and non-polyp 144, etc.).

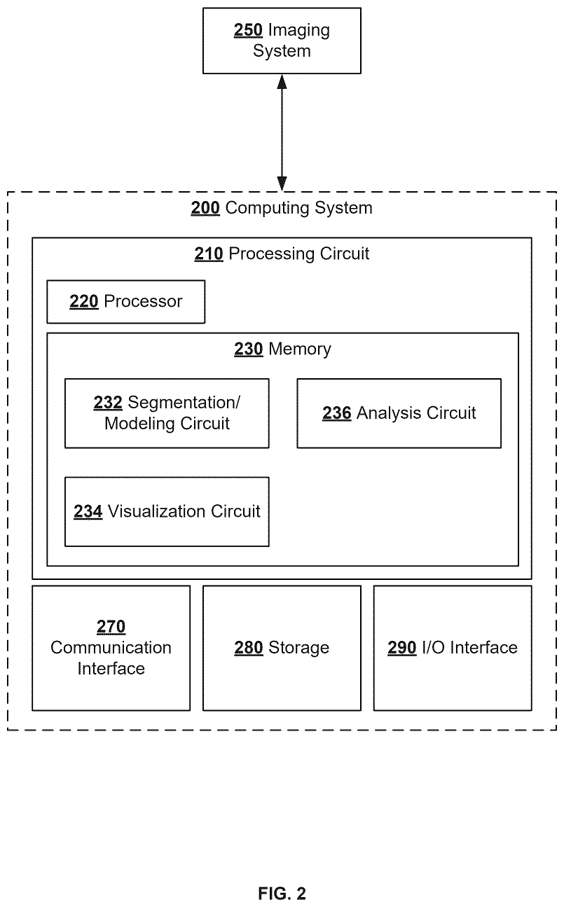

Referring to FIG. 2, computing system 200 is shown, according to an exemplary embodiment. Computing system 200 may perform one or more of the methods disclosed herein. For example, computing system 200 may segment image data, generate a 3D model based on the segmented image data, generate visualization(s) based on the 3D model to facilitate medical professional review, and/or automatically analyze the 3D model to identify features such as polyps within a region of interest such as a subject's colon. Computing system 200 may include processing circuit 210, communication interface 270, storage 280, and/or I/O interface 290. In various embodiments, computing system 200 is communicably connected to imaging system 250. Imaging system 250 may acquire and/or store one or more images for analysis. For example, imaging system 250 may include a computed tomography (CT) scanner that generates CT scan images of a subject's colon (e.g., mid region, etc.). However, it should be understood that imaging system 250 may include any other medical imaging system (e.g., an electroencephalograph system, a magnetoencephalography system, an electrocardiogram system, an x-ray system, a magnetic resonance imaging system, an ultrasound system, a magnetic particle imaging system, and/or the like).

Processing circuit 210 may include processor 220 and/or memory 230. Processor 220 may be a general purpose or specific purpose processor, an application specific integrated circuit (ASIC), one or more field programmable gate arrays (FPGAs), a group of processing components, or other suitable processing components. Processor 220 is configured to execute computer code or instructions stored in memory 230 or received from other computer readable media (e.g., CDROM, network storage, a remote server, etc.). Memory 230 may include one or more devices (e.g., memory units, memory devices, storage devices, and/or other computer-readable media) for storing data and/or computer code for completing and/or facilitating the various processes described in the present disclosure. Memory 230 may include random access memory (RAM), read-only memory (ROM), hard drive storage, temporary storage, non-volatile memory, flash memory, optical memory, or any other suitable memory for storing software objects and/or computer instructions. Memory 230 may include database components, object code components, script components, or any other type of information structure for supporting the various activities and information structures described in the present disclosure. Memory 230 may be communicably connected to processor 220 via processing circuit 210 and may include computer code for executing (e.g., by processor 220) one or more of the processes described herein. For example, memory 230 may have instructions stored thereon that, when executed by processor 220, cause processing circuit 210 to (i) receive image data, (ii) segment the image data and/or generate a 3D model based on the image data, (iii) detect one or more features (e.g., polyps, etc.) within the image data (or a model generated therefrom, etc.), and/or (iv) generate/present a user interface that surfaces otherwise unknown information (e.g., the one or more features, etc.) for a medical professional.

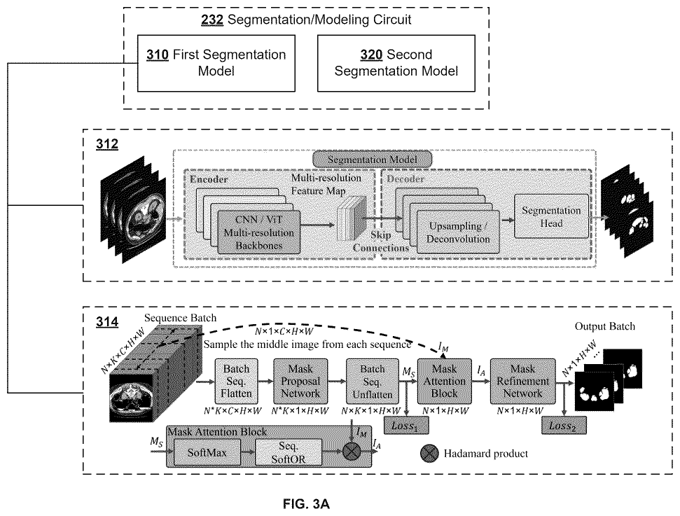

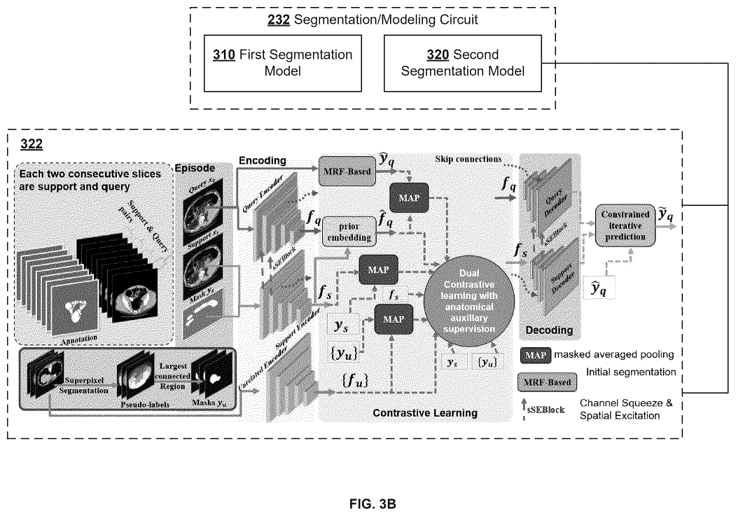

In various embodiments, memory 230 includes segmentation/modeling circuit 232, visualization circuit 234, and/or analysis circuit 236. Segmentation/modeling circuit 232 may segment image data (e.g., isolate a region of interest such as colon tissue/lumen within the image data) and/or generate a 3D model based on the image data (e.g., a 3D model of an ROI such as a subject's colon, etc.). Segmentation/modeling circuit 232 is discussed in greater detail with reference to FIGS. 3A-4.

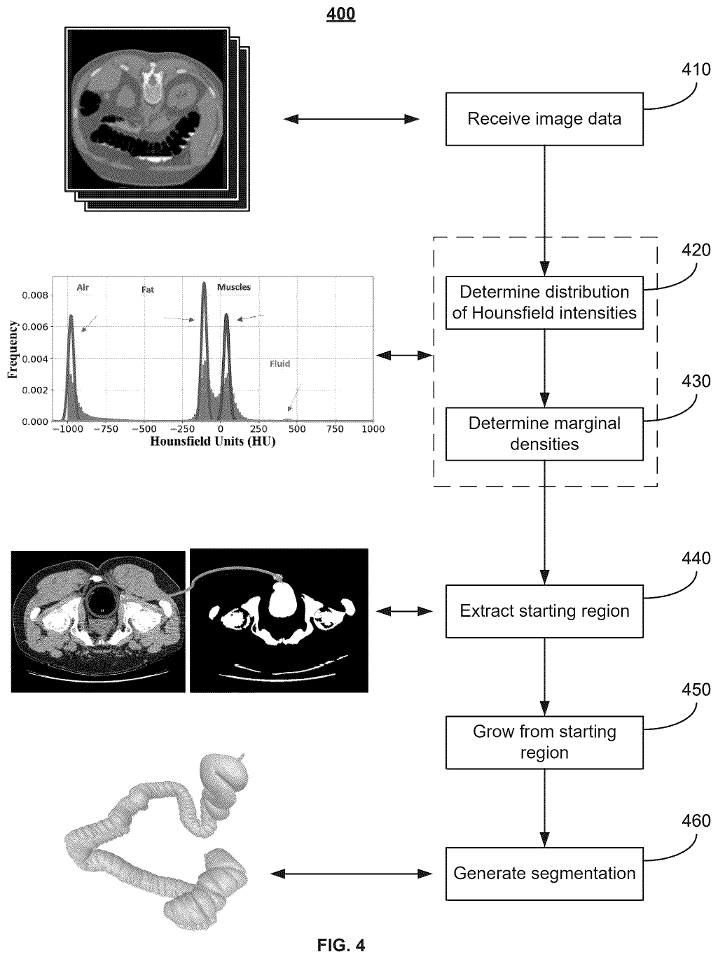

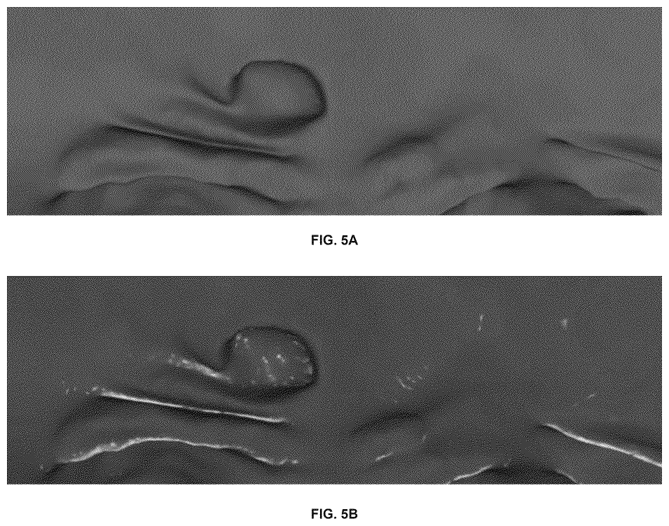

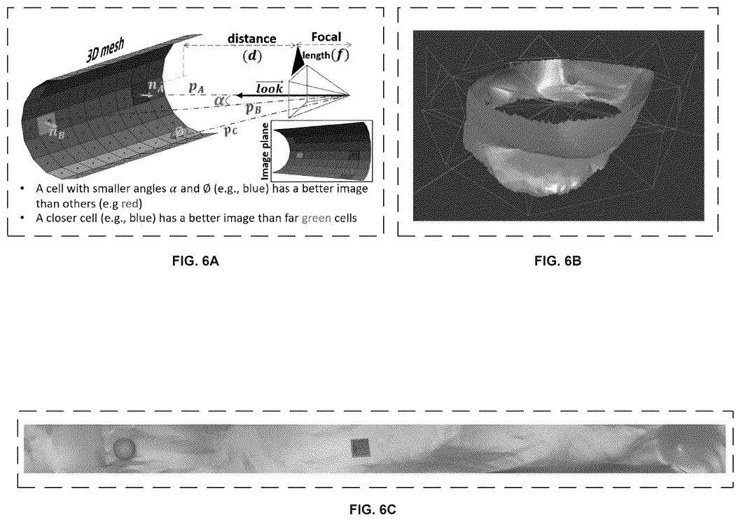

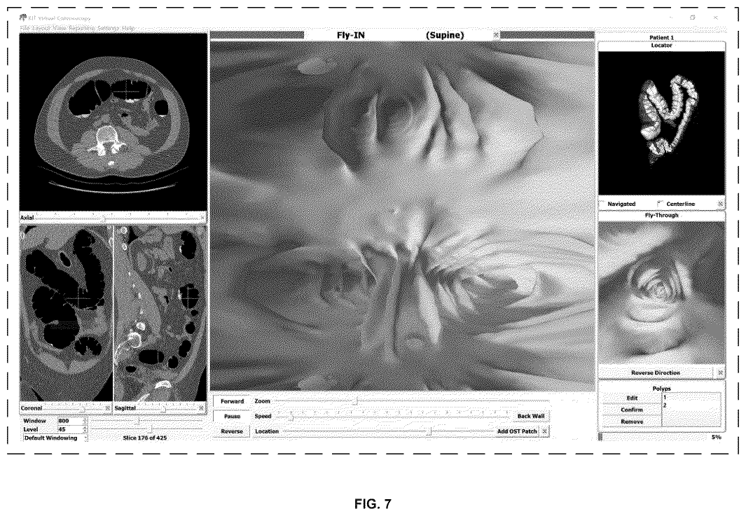

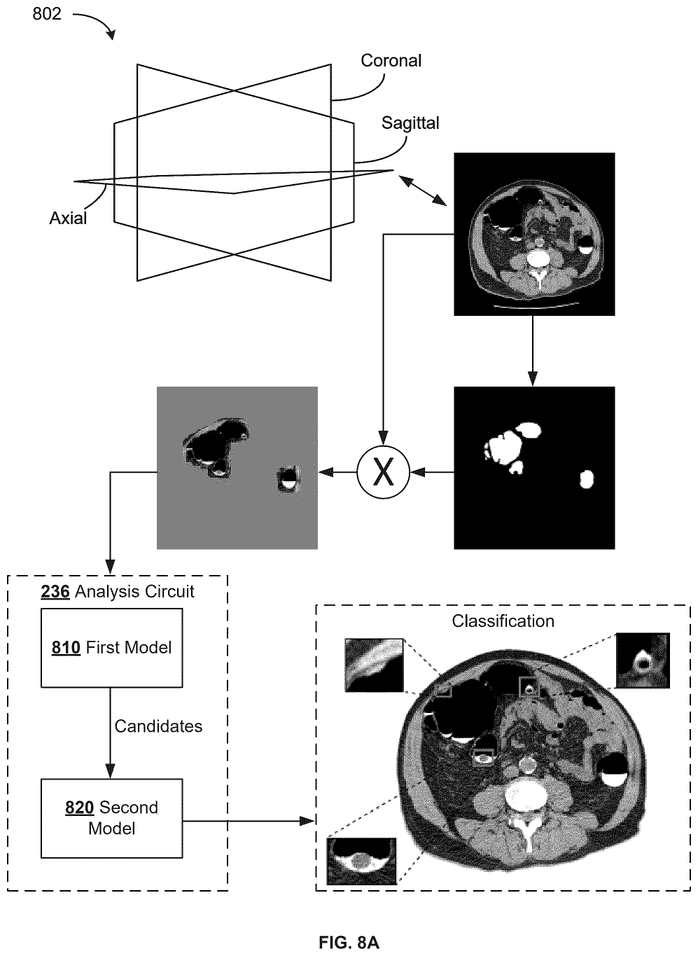

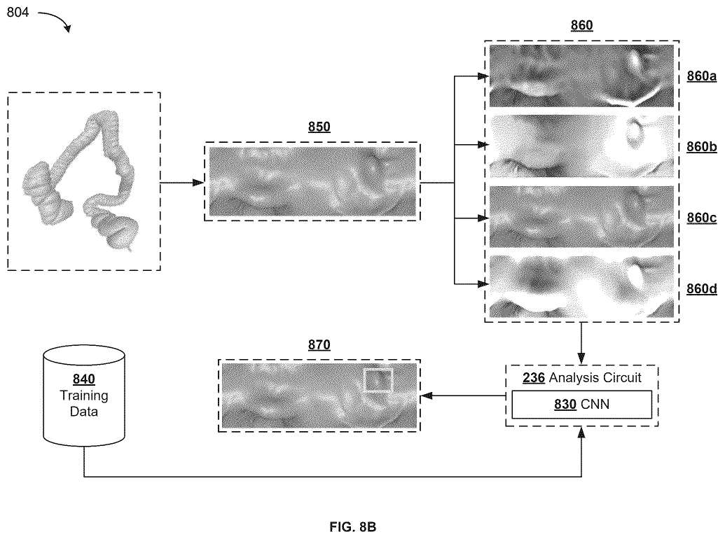

Visualization circuit 234 may generate one or more user interfaces to facilitate review by a healthcare professional. In various embodiments, visualization circuit 234 fuses 2D projections from a fly-in (FI) view and 3D representations in a virtual display (e.g., a virtual colonoscopy display). In various embodiments, visualization circuit 234 generates a “filet”-like 2D image of an internal surface of a subject (e.g., a colon ring). An example of this “filet”-like 2D image is shown in FIG. 6C. In various embodiments, visualization circuit 234 augments the 2D image with 3D information (e.g., curvature information). For example, as shown in FIGS. 5A-B, visualization circuit 234 may add a curvature value to each point on a colon surface represented as an RGB value (e.g., where the top image illustrates a virtual RGB image generated by visualization circuit 234 and the bottom image illustrates added curvature information highlighting convex and concave regions). Visualization circuit 234 is discussed in greater detail with reference to FIGS. 6A-7. Analysis circuit 236 may identify one or more features within image data. For example, analysis circuit 236 may automatically identify and characterize polyps in CT scan images of a subject's abdomen. In some embodiments, analysis circuit 236 implements a RetinaNet model using a focal loss function defined as:

$$ℒ ( ρ ) - {( 1 - y ρ - ( \frac{1 - y}{2} ) )}^{y} log ( y ρ ( \frac{1 - y}{2} ) )$$

where y is a tunable focusing parameter greater than or equal to zero. Analysis circuit 236 is discussed in greater detail with reference to FIGS. 8A-B.

Communication interface 270 may facilitate communication with one or more systems/devices. For example, computing system 200 may communicate via communication interface 270 with imaging system 250 and/or the like. Communication interface 270 may be or include wired or wireless communications interfaces (e.g., jacks, antennas, transmitters, receivers, transceivers, wire terminals, etc.) for conducting data communications with external systems or devices. In various embodiments, communication via communication interface 270 is direct (e.g., local wired or wireless communications). Additionally or alternatively, communications via communication interface 270 may utilize a network (e.g., a WAN, the Internet, a cellular network, etc.).

Storage 280 may store data/information associated with the various methods/operations described herein. For example, storage 280 may store model weights, image data, and/or the like. Storage 280 may be and/or include one or more memory devices (e.g., hard drive storage, temporary storage, non-volatile memory, flash memory, optical memory, and/or any other suitable memory device).

I/O interface 290 may facilitate input/output operations. For example, I/O interface 290 may include a display capable of presenting information to a user and an interface capable of receiving input from the user. In some embodiments, I/O interface 290 includes a display device configured to present a GUI to a user. I/O interface 290 may include hardware and/or software components. For example, I/O interface 290 may include a physical input device (e.g., a mouse, a keyboard, a touchscreen device, etc.) and software to enable the physical input device to communicate with computing system 200 (e.g., firmware, drivers, etc.). In some embodiments, I/O interface 290 includes an API to facilitate interaction with external systems (e.g., imaging system 250, etc.). For example, a user may use I/O interface 290 to access computing system 200 to analyze CT images acquired by imaging system 250.

Referring to FIGS. 3A-B, segmentation/modeling circuit 232 is shown, according to an exemplary embodiment. Segmentation/modeling circuit 232 may include first segmentation model 310 and/or second segmentation model 320. In some embodiments, first segmentation model 310 and second segmentation model 320 are integrated into a single model.

First segmentation model 310 may be and/or include first model 312 and/or second model 314. First model 312 may include an encoder and a decoder. In various embodiments, the encoder generates multiresolution feature maps that are passed to the decoder via skip connections. In various embodiments, the decoder fuses these features through upsampling/deconvolution blocks to generate a final feature map. In various embodiments, a segmentation head may use the feature map to generate a predicted segmentation mask. In various embodiments, first model 312 is trained using custom dice (e.g., by using the predicted and ground truth masks to update the network weights via back propagation).

Second model 314 may include (i) a batch-sequence flatten (BSF) block, (ii) a mark proposal network (MPN) block, (iii) a batch-sequence unflatten (BSU) block, (iv) a mask attention (MA) block, and/or (v) a mask refinement network (MRN) block. In various embodiments, second model 314 includes one or more loss functions. In various embodiments, the BSF block receives a batch of images (e.g., CT image sequences, etc.) I, as an input, with size N×K×C×H×W, where N is the batch size, K is the sequence length, C is the number of channels, W is the image width, and H is the image height. In various embodiments, the BSF block converts the batch of images to another batch with an equivalent size of N*K to prepare it for the MPN block.

In various embodiments, the MPN block accepts the flattened batch and generates a batch of the corresponding proposed masks, which may be converted back to a batch of mask sequences M_{S} by the BSU block. In various embodiments, the MA block receives proposed mask sequences, which are converted into probabilities (e.g., via a soft-max layer). In various embodiments, the MPN block transforms each mask sequence into a single attention mask corresponding to the middle image of the sequence (e.g., by summing all masks per sequence pixel-wise, etc.). In various embodiments, the middle image from each sequence is sampled and attended by its corresponding attention mask using a Hadamard product to produce a batch of attended images I_{A}.

In various embodiments, the MRN block receives the attended batch of images I_{A} and generates a final corresponding batch of segmented masks. In various embodiments, the MPN and MRN blocks are and/or include an OTS 2D-segmentation model. In various embodiments, second model 314 implements a first loss function to force the MPN block to propose accurate masks and/or a second loss function to force the MRN block to generate accurate segmentation masks from the attended images I_{A}. In some embodiments, the loss function is defined as:

$$ℒ ( x , y , b ) = α \cdot DiL ( x , y ) + ( 1 - α ) \cdot BL ( x , b )$$

where x represents the predicted segmentation mask, y represents the ground truth binary mask, b represents the computed boundary map, α is a hyperparameter (e.g., boundary weight) controlling the trade-off between the dice loss (DiL) and the boundary loss (BL). In various embodiments, DiL is defined by:

$$DiL = 1 - \frac{2 {\sum }_{i = 1}^{N} {p}_{i} {g}_{i}}{{\sum }_{i = 1}^{N} {p}^{2} + {\sum }_{i = 1}^{N} {g}^{2}}$$

Where pᵢ∈[0,1] represents the probability for the i-th pixel to be ROI (e.g., colon), gᵢ∈{0,1} represents the ground truth for the same pixel, and N represents the total number of pixels. In various embodiments, BL is defined as:

$$B L ( x , b ) = \frac{1}{N} {\sum }_{i = 1}^{N} {b}_{i} \cdot {x}_{i}$$

where N represents the total number of pixels, bᵢ represents the value of the boundary map, and xi represents the value of the predicted segmentation mask at pixel i. In various embodiments, the boundary map b is determined by finding the minimum Euclidean distance from each pixel (i,j) to any pixel on the boundary of the binary mask M. In various embodiments, the distance transform assigns a higher value to pixels closer to the boundary and a lower value to pixels in the interior of the binary mask.

In various embodiments, second model 314 applies a higher weight to pixels near the boundary of the ROI (e.g., colon). For example, for each CT image pixel, second model 314 may assign a weight based on a weight map generated from each ground truth segmentation mask corresponding to the slice

Referring specifically to FIG. 3B, second segmentation model 320 may include model 322. As a high-level overview, model 322 may (i) use consecutive image slices as support and query pairs, (ii) randomly sample negative samples from slices lacking a ROI (e.g., colon) to improve feature discriminability, (iii) integrate an initial segmentation from a Markov Randon Field (MRF)-based algorithm, (iv) apply masked average pooling (e.g., to extract features, etc.), (v) applying dual contrastive learning (DCL) to create an embedding space, and (vi) generating a final segmentation by iteratively refining the initial segmentation with decoders and skip connections. In various embodiments, model 322 operates on 2D image slices while effectively incorporating 3D contextual information (e.g., due to the sequential dependency), thereby enhancing segmentation accuracy without requiring the computational complexity associated with a fully 3D network (e.g., a 3D-CNN).

In various embodiments, model 322 performs an MRF-based segmentation algorithm including (i) Gaussian fitting using an expectation-maximization algorithm, (ii) region growing (e.g., using an identified starting feature such as a rectum as a seed for extracting additional features), (iii) application of a graph cut algorithm with the region growing as a seed to generate an initial segmentation. Model 322 may define a support set

$$S = \{ {x}_{s}^{c} , {y}_{s}^{c} \}$$

and a query set

$$Q = \{ {x}_{q}^{c} , {y}_{q}^{c} \}$$

for a set of images X and its corresponding set of binary masks Y, where

$${x}_{s ( q )}^{c} ∈ X , {y}_{s ( q )}^{c} ∈ Y$$

and c represents an arbitrary class in a set of classes C. For example, c may represent a colon class and c̅ may represent a non-colon class. In various embodiments, model 322 implements episodic training under supervision. Additionally or alternatively, model 322 may incorporate unrelated slices

$$U = \{ {x}_{u}^{\overline{c}} , {y}_{u}^{\overline{c}} \}$$

(e.g., which may be rich in other anatomical structures, thereby enhancing discriminability, etc.). In various embodiments, model 322 organizes input image data (e.g., CT scans, etc.) into pairs of consecutive slices. For each episode, model 322 may randomly select three negative samples from slices that do not contain the ROI (e.g., colon, etc.) and may apply an unsupervised GC-based algorithm to generate pseudo-labels

$$\{ {y}_{u}^{\overline{c}} \} .$$

In various embodiments, model 322 passes the episode

$$( {x}_{s}^{c} , {x}_{q}^{c} , \{ {x}_{u}^{\overline{c}} \} )$$

through an encoder (e.g., a sSENet's, etc.) to extract features (fₛ, f_{q}, {fᵤ}). In various embodiments, model 322 integrates the features and their corresponding masks to construct an embedding space that attracts

$$( {x}_{s}^{c} , {x}_{q}^{c} )$$

while repelling

$$( {x}_{q}^{c} , {x}_{u}^{\overline{c}} ) ,$$

thereby optimizing the feature representation (e.g., via a AAS-DCL scheme, etc.). In some embodiments, model 322 performs a few-shot segmentation (FSS).

In various embodiments, model 322 is trained via class-level prototypical contrastive learning. For example, computing system 200 may generate a colon prototype using a masked average pooling (MAP) operation and may use the colon prototype as a feature vector that encapsulates the distinctive characteristics of the colon across various CT slices. In various embodiments, model 322 differentiates between feature and non-feature structures (e.g., colon and non-colon structures) by comparing the query feature from an unseen image to the colon prototype (e.g., where non-target class features act as negative examples).

In various embodiments, model 322 computes the query prototype (e.g., via MAP) using the query initial mask ŷ_{q}. In some embodiments, model 322 computes the background prototype vᵤ (e.g., via MAP) using unrelated features and their corresponding masks. In various embodiments, model 322 iteratively refines the query prediction using a similarity consistency constraint (e.g., based on a similarity map between fₛ and f_{q}). During training, a cross-entropy loss may be used to compute a prediction error against the ground truth. The inference stage may begin by identifying a starting point (e.g., the rectum, etc.) and generating an initial mask for the colon. The starting point may serve as a support sample and its respective is a query. Model 322 may select three randomly unrelated slices and the support for the next slice in the sequence may be the segmented query slice (e.g., repeating/iterating until all colon regions have been segmented, etc.).

In various embodiments, a contrastive learning module of model 322 is trained using a infoNCE loss [symbol] (v_{q}, vₛ, vᵤ) according to:

$$ℒ ( {v}_{q} , {v}_{s} , {v}_{u} ) = - {v}_{q} \cdot \frac{{v}_{s}}{τ} + log {\sum }_{i = 1}^{n} exp ( {v}_{q} \cdot \frac{{v}_{u i}}{τ} )$$

where τ is a control parameter, n is a number of negative samples, v_{q} is the query prototype, vₛ is the support prototype, and vᵤ is the background prototype. In various embodiments, the prototypes are generated by global average pooling of features and corresponding masks. In various embodiments, computing system 200 generates support features {fₛ} and their corresponding masks {yₛ} via a masked average pooling (MAP) operation:

$${v}_{s} = \frac{{\sum }_{r} {y}_{s} ( r ) \cdot {f}_{s} ( r )}{{\sum }_{r} {y}_{s} ( r )}$$

In various embodiments, model 322 uses the query initial mask \hat{f}_{q} to generate the query prototype.

Referring now to FIG. 4, method 400 is shown, according to an exemplary embodiment. In various embodiments, method 400 segments one or more images into different regions of interest (ROI). For example, method 400 may identify which portions of each image correspond to a subject's colon and may mask out the rest of the image. At step 410, method 400 includes receiving image data. The image data may include one or more computed tomography (CT) scan images/slices. For example, the image data may include a DICOM file having volumetric representation and/or a number of CT scan slices from one or more views (e.g., sagittal, coronal, axial, etc.).

At steps 420-430, method 400 may include determining image components. For example, method 400 may include determining which portions of the image data correspond to air, fat, muscle, and/or fluid. At step 420, method 400 includes determining the distribution of Hounsfield intensities within the image data. For example, step 420 may include calculating the empirical distribution of Hounsfield intensities in a DICOM volume. At step 430, method 400 includes determining the marginal densities of one or more components. For example, step 430 may include determining the marginal densities of air, fat, muscle, and fluid by fitting four Gaussian components using an expected maximization (EM) algorithm. In various embodiments, the peak of air is around −1000 HU and the peak of fluid is greater than 300 HU. In various embodiments, steps 420-430 include identifying one or more colon regions. For example, method 400 may include identifying regions based on one or more HU thresholds. To continue the example, method 400 may include identifying portions of an image slice having an HU value less than t₁ and greater than t₂ as colon regions, where t₁ is the threshold between air and fat and t₂ is the threshold between muscle and fluid. In some embodiments, method 400 includes labeling a volume using a grey-level probabilistic model.

At step 440, method 400 includes extracting a starting region. In various embodiments, the starting region is the rectum. For example, method 400 may include identifying the rectum in the initial segmentation by identifying a disk-like region that has a low HU. In various embodiments, the starting region is used as a seed from which other image regions (e.g., colon regions) are extracted.

At step 450, method 400 includes region growing. For example, method 400 may include extracting the colon region from each CT scan slice via region growing starting from the starting region. In various embodiments, the region growing is restricted region growing performed using the morphological operation (e.g., to facilitate separation between tissue/non-tissue classes). In various embodiments, method 400 includes creating a weighted undirected graph with vertices corresponding to the set of volume voxels [symbol] and a set of edges connecting these vertices. In various embodiments, method 400 includes minimizing the function:

$$E ( f ) = {\sum }_{\{ i , j \}}^{N} V ( {f}_{i} , {f}_{j} ) + {\sum }_{i ∈ 𝒫} D ( {f}_{i} )$$

where D(fᵢ) measures how much assigning a label f; to voxel i disagrees with the voxel intensity Iᵢ (e.g., which can be determined from the log-likelihood of each class), and [symbol] is a neighborhood system of unordered pairs {i, j} of neighboring voxels in [symbol] .

At step 460, method 400 includes generating a final segmentation. For example, method 400 may include using the output of step 450 as a seed for a graph cut algorithm. In various embodiments, the output of step 460 is a 3D model of an organ (e.g., a colon). Additionally or alternatively, the output of step 450 may include one or more masks (e.g., binary masks, etc.) that identify specific tissue regions (e.g., corresponding to a subject's colon, etc.) within each image slice. In some embodiments, method 400 includes refining the masks. For example, method 400 may include dilating the segmented regions within the mask (e.g., to ensure that tissue such as a colon wall is included in the segmented regions). In various embodiments, the masks are used to focus the detection system on a specific tissue region (e.g., the colon). For example, the image data may be multiplied by the masks to produce masked image data that is used as an input to the detection system.

Referring to FIGS. 6A-C, an example method of generating a visualization and two example visualizations are shown. In various embodiments, visualization circuit 234 performs the method shown in FIG. 6A and generates the user interface elements shown in FIGS. 6B-C. For example, visualization circuit 234 may perform 3D reconstruction to generate a surface representation of an ROI (e.g., a subject's colon) and may identify a centerline of the ROI. In various embodiments, visualization circuit 234 projects surface cells onto an image plane (e.g., minimizing local deformations and/or losses, etc.). In various embodiments, visualization circuit 234 models visualization loss as a function of (i) an angle (α) between a projection direction (p) and a camera's principal axis (\vec{look}), (ii) an angle (Φ) between the projection direction (p) and the cell's normal vector (n), and (iii) a ratio between the camera focal length (f) and the cell's distance (d) to the projection center on the direction of \vec{look}. As shown in FIG. 6B, eight virtual cameras may be used in a ring to generate a distortionless filet of an ROI. In various embodiments, visualization circuit 234 generates one or more images coding geometric surface features based on the model of the ROI. For example, visualization circuit 234 may generate (i) a surface curvature map (e.g., by determining the curvature using the algebraic set point surface, where the curvature is based on moving least squares fitting algebraic spheres to the surface, etc.), (ii) a normal map (e.g., by taking the cross product of two vectors on the surface and for each vertex on the surface, subtracting the 3D coordinates (x, y, z) for the vertex from two neighbors 3D coordinates, etc.), (iii) a depth map (e.g., that reflects the smallest distance between each surface point and the centerline). In various embodiments, the normal map is represented as a three-channel (e.g., RGB) image to represent the normal vector (Nₓ, N_{y}, N_{z}). Examples of images coding geometric surface features are shown in FIG. 8B.

Referring to FIG. 7, an example user interface is shown. In various embodiments, visualization circuit 234 generates the user interface. For example, visualization circuit 234 may generate desk-like rig of virtual cameras including a fly-in view, a locator view, a fly-through view, and one or more views of the underlying medical imaging (e.g., axial, coronal, and/or sagittal CT scan slices, etc.). In various embodiments, visualization circuit 234 generates a 3600 visualization of an ROI, which, when projected, provides a “filet”-like display of the internal surface of the ROI.

Referring now to FIG. 8A, method 802 of performing feature (e.g., polyp) detection using analysis circuit 236 is shown, according to an exemplary embodiment. Analysis circuit 236 may include first model 810 and second model 820. In various embodiments, first model 810 is and/or includes a convolutional neural network (CNN). For example, first model 810 may include a You Only Look Once (YOLO) model. However, it should be understood that other models may be used (e.g., a Faster-RCNN model with Resnet, a Retina Net model with Efficient Net backbone, a sparse RCNN model, a swim transformer model, etc.). In various embodiments, first model 810 identifies features (e.g., polyps) from a first view of received image data. For example, first model 810 may identify polyps from an axial view. In various embodiments, a confidence score threshold may be used to tune the sensitivity of first model 810. In various embodiments, first model 810 receives segmented images as an input (e.g., CT scan slices having a binary mask applied to highlight an ROI such as a subject's colon within the slices). In the context of polyp detection, the ROI may include a subject's colon.

In various embodiments, second model 820 is and/or includes a multi-view fusion network (MVN). Second model 820 may use three 2D images to validate each candidate feature (e.g., polyp, etc.) identified by first model 810. Validating each candidate feature may reduce the number of false positives. In various embodiments, analysis circuit 236 requires less time and memory compared with other models that are trained on volume data (e.g., 3D-CNNs, LSTM networks, etc.). In some embodiments, second model X30 implements a Markov chain model. For example, second model 820 may calculate:

$$P ( Y \vert X ) = P ( {Y}_{c} \vert X , {Y}_{s} , {Y}_{a} ) P ( {Y}_{s} \vert X , {Y}_{a} ) P ( {Y}_{a} \vert X )$$

where X={X_{c}, Xₛ, Xₐ} is the input sequence of the coronal, sagittal, and axial views and Y={Y_{c}, Yₛ, Yₐ} is the predicted output sequence. In various embodiments, second model 820 may determine the predicted output sequence as shown below:

$$P ( {Y}_{a} \vert X ) = Sigmoid ( FC ( {f}_{a} ) )$$

$$P ( {Y}_{s} \vert X , {Y}_{a} ) = Sigmoid ( FC ( [ {f}_{s} , {h}_{a} , {y}_{a} ] ) )$$

$$P ( {Y}_{c} \vert X , {Y}_{a} , {Y}_{s} ) = Sigmoid ( FC ( [ {f}_{c} , {h}_{s} , {y}_{s} , {y}_{a} ] ) )$$

where FC(⋅) is a fully connected network.

In various embodiments, analysis circuit 236 is trained on annotated images. For example, analysis circuit 236 may be trained on CT scan images from supine and prone subjects. In various embodiments, the images include annotations identifying polyps within the images. In some embodiments, the training data is augmented. For example, analysis circuit 236 may apply one or more transformations (e.g., flipping the images, adjusting exposure, saturation, and/or brightness, etc.) to the annotated images before training. In various embodiments, first model 820 is trained on axial views and second model 820 is trained on one or more of axial, coronal, and/or sagittal views. In various embodiments, once trained, analysis circuit 236 has a sensitivity of greater than 85% and a mean average precision (mAP) of at least 80%. For example, analysis circuit 236 may have a sensitivity of 95% and an area under the curve (AUC) of 95% (e.g., indicating that analysis circuit 236 rejects most false positives). In various embodiments, analysis circuit 236 requires less memory and processing power than other models. For example, analysis circuit 236 may have fewer than one-tenth the number of parameters of other classifiers having similar or worse performance. In various embodiments, second model 820 generates a classification (e.g., polyp, not-polyp, etc.). Additionally or alternatively, second model 820 may identify a location of the feature within one or more images (e.g., using a bounding box, etc.). In some embodiments, analysis circuit 236 generates a confidence score associated with each feature. In some embodiments, analysis circuit 236 determines characteristics of the detected feature. For example, analysis circuit 236 may determine a size of a detected polyp.

Referring now to FIG. 8B, method 804 of performing feature (e.g., polyp) detection using analysis circuit 236 is shown, according to an exemplary embodiment. In some embodiments, method 804 is different than method 802 (e.g., may use different models and/or different inputs, etc.). In some embodiments, methods 802 and 804 are integrated into a single method/system. As a high-level example, method 804 may include (i) receiving a model (e.g., a 3D model) of a subject's colon, (ii) generating a 2D image of an ROI within the subject's colon (shown as image 850) using one or more virtual cameras and the model, (iii) generating one or more augmented images (shown as augmented images 860) based on image 850, and (iv) analyzing augmented images 860 using analysis circuit 236 to detect one or more features such as polyps and characterizing the size and location of the one or more features (shown in display 870). In various embodiments, augmented images 860 include curvature map 860a, normal map 860b, fly-in visualization albedo/lightning image 860c, and/or depth map 860d.

In various embodiments, analysis circuit 236 includes CNN 830. CNN 830 may be trained using training data from a database (shown as training data 840). In various embodiments, training data 840 includes 3D surface information from virtual colonoscopy images and/or geometric features generated therefrom (e.g., where the features encode 3D surface geometry). In various embodiments, CNN 830 receives (i) 2D images generated from the 3D model of the subject's colon and (ii) 3D geometric feature maps (e.g., depth and curvature maps). In some embodiments, systems and methods of the present disclosure combine the 2D images and the 3D geometric feature maps in multi-channel images for analysis via analysis circuit 236.

Systems and methods of the present disclosure may facilitate identifying one or more features within image data. For example, systems and methods of the present disclosure may facilitate automated detection of colorectal cancer (CRC) or symptoms associated therewith (e.g., colon polyps, etc.). In various embodiments, inference using systems and methods of the present disclosure significantly reduce a number of floating-point operations (FLOPs) required to perform feature detection from medical image data. For example, systems and methods of the present disclosure may provide a 70× reduction in FLOPs (e.g., due, at least in part, to a 48× reduction in the number of network parameters required, etc.). It should be understood that while detection of polyps is used throughout the disclosure as an example, systems and methods of the present disclosure may be applied to detection of other features as well. As used herein, a “polyp” is a growth attached to the luminal wall of a colon and/or rectum.

As utilized herein with respect to numerical ranges, the terms “approximately,” “about,” “substantially,” and similar terms generally mean+/−10% of the disclosed values, unless specified otherwise. As utilized herein with respect to structural features (e.g., to describe shape, size, orientation, direction, relative position, etc.), the terms “approximately,” “about,” “substantially,” and similar terms are meant to cover minor variations in structure that may result from, for example, the manufacturing or assembly process and are intended to have a broad meaning in harmony with the common and accepted usage by those of ordinary skill in the art to which the subject matter of this disclosure pertains. Accordingly, these terms should be interpreted as indicating that insubstantial or inconsequential modifications or alterations of the subject matter described and claimed are considered to be within the scope of the disclosure as recited in the appended claims.

It should be noted that the term “exemplary” and variations thereof, as used herein to describe various embodiments, are intended to indicate that such embodiments are possible examples, representations, or illustrations of possible embodiments (and such terms are not intended to connote that such embodiments are necessarily extraordinary or superlative examples).

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

References herein to the positions of elements (e.g., “top,” “bottom,” “above,” “below”) are merely used to describe the orientation of various elements in the figures. It should be noted that the orientation of various elements may differ according to other exemplary embodiments, and that such variations are intended to be encompassed by the present disclosure.

The present disclosure contemplates methods, systems, and program products on any machine-readable media for accomplishing various operations. The embodiments of the present disclosure may be implemented using existing computer processors, or by a special purpose computer processor for an appropriate system, incorporated for this or another purpose, or by a hardwired system. Embodiments within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. Combinations of the above are also included within the scope of machine-readable media. Machine-executable instructions include, for example, instructions and data which cause a general-purpose computer, special purpose computer, or special purpose processing machines to perform a certain function or group of functions.

Although the figures and description may illustrate a specific order of method steps, the order of such steps may differ from what is depicted and described, unless specified differently above. Also, two or more steps may be performed concurrently or with partial concurrence, unless specified differently above. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

The term “client or “server” include all kinds of apparatus, devices, and machines for processing data, including by way of example a programmable processor, a computer, a system on a chip, or multiple ones, or combinations, of the foregoing. The apparatus may include special purpose logic circuitry, e.g., a field programmable gate array (FPGA) or an application specific integrated circuit (ASIC). The apparatus may also include, in addition to hardware, code that creates an execution environment for the computer program in question (e.g., code that constitutes processor firmware, a protocol stack, a database management system, an operating system, a cross-platform runtime environment, a virtual machine, or a combination of one or more of them). The apparatus and execution environment may realize various different computing model infrastructures, such as web services, distributed computing and grid computing infrastructures.

The systems and methods of the present disclosure may be completed by any computer program. A computer program (also known as a program, software, software application, script, or code) may be written in any form of programming language, including compiled or interpreted languages, declarative or procedural languages, and it may be deployed in any form, including as a stand-alone program or as a module, component, subroutine, object, or other unit suitable for use in a computing environment. A computer program may, but need not, correspond to a file in a file system. A program may be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program may be deployed to be executed on one computer or on multiple computers that are located at one site or distributed across multiple sites and interconnected by a communication network.

The processes and logic flows described in this specification may be performed by one or more programmable processors executing one or more computer programs to perform actions by operating on input data and generating output. The processes and logic flows may also be performed by, and apparatus may also be implemented as, special purpose logic circuitry (e.g., an FPGA or an ASIC).

Processors suitable for the execution of a computer program include, by way of example, both general and special purpose microprocessors, and any one or more processors of any kind of digital computer. Generally, a processor will receive instructions and data from a read only memory or a random-access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data (e.g., magnetic, magneto-optical disks, or optical disks). However, a computer need not have such devices. Moreover, a computer may be embedded in another device (e.g., a vehicle, a Global Positioning System (GPS) receiver, etc.). Devices suitable for storing computer program instructions and data include all forms of non-volatile memory, media and memory devices, including by way of example semiconductor memory devices (e.g., EPROM, EEPROM, and flash memory devices; magnetic disks, e.g., internal hard disks or removable disks; magneto-optical disks; and CD ROM and DVD-ROM disks). The processor and the memory may be supplemented by, or incorporated in, special purpose logic circuitry.

To provide for interaction with a user, implementations of the subject matter described in this specification may be implemented on a computer having a display device (e.g., a CRT (cathode ray tube), LCD (liquid crystal display), OLED (organic light emitting diode), TFT (thin-film transistor), or other flexible configuration, or any other monitor for displaying information to the user. Other kinds of devices may be used to provide for interaction with a user as well; for example, feedback provided to the user may be any form of sensory feedback (e.g., visual feedback, auditory feedback, or tactile feedback).

Implementations of the subject matter described in this disclosure may be implemented in a computing system that includes a back-end component (e.g., as a data server), or that includes a middleware component (e.g., an application server), or that includes a front end component (e.g., a client computer) having a graphical user interface or a web browser through which a user may interact with an implementation of the subject matter described in this disclosure, or any combination of one or more such back end, middleware, or front end components. The components of the system may be interconnected by any form or medium of digital data communication (e.g., a communication network). Examples of communication networks include a LAN and a WAN, an inter-network (e.g., the Internet), and peer-to-peer networks (e.g., ad hoc peer-to-peer networks).

What is claimed is:

1. A method for colon polyp detection, the method comprising:
determining, by a first classifier, wherein the first classifier comprises a first CNN model configured to use a first set of medical images comprising segmented medical images of a colon, a candidate polyp location and size; and
classifying, by a second classifier, wherein the second classifier comprises a second CNN model configured use a second set of medical images of the colon, a candidate polyp as a polyp or not a polyp;
wherein the first classifier and the second classifier are configured in a cascade.

2. The method of claim 1, wherein the first CNN model is trained on the first set of medical images of the colon.

3. The method of claim 2, wherein the first set of medical images comprises segmented CT scans in an axial view of the colon.

4. The method of claim 3, wherein the second CNN model is trained on a second set of medical images of the colon.

5. The method of claim 4, wherein the second set of medical images comprises 2D images in an axial view, a sagittal view, and a coronal view of the colon.

6. The method of claim 5, wherein the second CNN model is trained independently on each of the axial view, sagittal view, and coronal view of the 2D images.

7. The method of claim 6, wherein the second CNN model is configured to provide a classification and a weight related to each of the axial view, sagittal view, and coronal view of the 2D images.

8. The method of claim 7, wherein the trained second CNN model is configured to select and output a classification prediction based on the classification and the weight related to each of the axial view, sagittal view, and coronal view of the 2D images.

9. The method of claim 8, wherein the second set of medical images comprise DICOM data.

10. The method of claim 9, wherein the first classifier is configured to predict all true positive candidate polyps and a number of false positives candidate polyps.

11. The method of claim 10, wherein the second classifier is applied to each candidate polyp location and size.

12. The method of claim 11, wherein the first CNN model and the second CNN model are 2D CNN networks.

13. A system for colon polyp detection, the system comprising a processor and a memory, the memory storing instructions thereon, that when executed by the processor, cause the processor to:
receive a set of CT scans, wherein a colon is segmented from the set of CT scans;
construct a 3D model of the colon from the set of CT scans;
simulate an inner wall of a region of interest of the colon;
generate 2D images of the simulated inner wall;
generate a plurality of feature maps using the 2D images and the 3D model; and
detect, by a convolutional neural network (CNN) model using the plurality of feature maps, a candidate polyp location and size.

14. The system of claim 13, wherein detecting the candidate polyp location and size comprises detecting the candidate polyp location and size by the CNN model.

15. The system of claim 14, wherein the plurality of feature maps encode 3D surface geometry.

16. The system of claim 15, wherein the plurality of feature maps are combined as multi-channel images and provided to the CNN model.

17. The system of claim 16, wherein the CNN model is trained and validated using the plurality of feature maps.

18. The system of claim 17, wherein the plurality of feature maps comprise a depth map, a normal map, and a curvature map.

19. The system of claim 18, wherein the curvature map comprises a curvature, wherein the curvature is calculated using moving least squares (MILS) fitting algebraic spheres to a surface of the 3D model of the colon.

20. A non-transitory computer-readable storage medium, having instructions stored thereon, that, when executed by a processor, cause the processor to:
receive image data comprising a plurality of CT scan images of at least a portion of a subject;
segment the CT scan images to identify portions of each image corresponding to the subject's colon;
analyze axial views of the segmented CT scan images to identify a candidate polyp using a first CNN;
analyze at least two of axial views, sagittal views, and coronal views CT scan images corresponding to the candidate polyp using a second model to classify the candidate polyp as a polyp or not a polyp; and
generate a user interface that includes the classified candidate polyp.
